(19) United States
(12) Patent Application Publication
(10) Pub. No.: US 2014/0072611 A1
(43) Pub. Date: Mar. 13, 2014

(54) Methods and Compositions for Treating Post-Cardial Infarction Damage

(71) Applicant: FIBROCELL TECHNOLOGIES, INC., EXTON, PA (US)

(72) Inventor: John M. Maslowski, Pottstown, PA (US)

(73) Assignee: FIBROCELL TECHNOLOGIES, INC., EXTON, PA (US)

(21) Appl. No.: 14/021,671

(22) Filed: Sep. 9, 2013

Related U.S. Application Data
(60) Provisional application No. 61/698,115, filed on Sep. 7, 2012.

(51) Int. Cl. A61K 35/12

(52) U.S. Cl. CPC A61K 35/33
USPC 424/443; 424/93.7

(57) ABSTRACT

Compositions for delaying, attenuating or preventing cardiac remodeling following cardiac injury contain fibroblast cells in a dosage providing an effective amount to delay, attenuate or prevent cardiac remodeling following cardiac injury. These cells are obtained by biopsy, preferably from the patient, then cultured and proliferated prior to use. It has been discovered that certain subpopulations of these cells are even better suited for repair or regeneration of tissue, the cells exhibiting properties similar to stem cells or multipotent cells. In a preferred embodiment, the cells are administered to delay, attenuate or prevent cardiac remodeling following cardiac injury.

### CROSS-REFERENCE TO RELATED APPLICATIONS

This application claims the benefit of U.S. Provisional Application No. 61/698,115, filed on Sep. 7, 2012. The entire disclosure of the above application is incorporated herein by reference.

### FIELD OF THE INVENTION

The present invention is generally in the field of cardiac tissue repair and regeneration by implantation or injection of cells that form cardiac tissue.

### BACKGROUND OF THE INVENTION

Myocardial infarction (MI), commonly known as a heart attack, is death of heart muscle that generally results from the sudden loss of blood supply to the heart tissue. The loss of blood supply often results from closure of the coronary artery or any other artery feeding the heart which nourishes a particular part of the heart muscle. The cause of this event is generally attributed to arteriosclerosis in coronary vessels, although it can also arise due to viral infection or other unknown causes. MI can result from a slow progression of closure of the blood vessel, from, for example, 95% then to 100%. However, MI can also be a result of minor blockages, where the flow of blood is blocked, for example, by rupture of a cholesterol plaque resulting in blood clotting within the artery. The resulting ischemia and ensuing oxygen shortage if left untreated for a sufficient length of time can cause death or damage of the heart muscle tissue (myocardium).

An important component in the progression to heart failure is remodeling of the heart due to mismatched mechanical forces between the infracted region and the healthy tissue, resulting in uneven stress and strain distribution in the left ventricle. If impaired blood flow to the heart lasts long enough, it triggers a process called the ischemic cascade, in which the heart cells die and do not regenerate. A collagen scar forms in place of the cardiomyocytes. Studies indicate that apoptosis may also play a role in the process of tissue damage subsequent to myocardial infarction (Krijnen, et al., J. Clin. Pathol., 55(11): 801-11 (2002)). As a result, the patient's heart can be permanently damaged. The scar tissue formed in the ischemic cascade also puts the patient at risk for potentially life threatening arrhythmias. The scar tissue is a hostile environment for cells due to its decreased blood flow and acidic pH. Scar tissue is also non-contractile, which reduces the overall cardiac output of the heart. Sutton, Circulation, 101(25):2981-8 (2000).

Once an MI occurs, remodeling of the heart begins. The term cardiac remodeling was initially coined to describe the prominent changes that occur after myocardial infarction. Pfeffer, et al., Circulation, 57:84-95 (1985). Cardiac remodeling involves molecular, cellular, and interstitial changes that manifest clinically as changes in size, shape, and function of the heart which occur after injury or stress stimulation. Ventricular remodeling involves progressive enlargement of the ventricle with depression of ventricular function. Myocyte function in the myocardium remote from the initial myocardial infarction becomes depressed. Ventricular remodeling usually occurs weeks to years after myocardial infarction. There are many potential mechanisms for ventricular remodeling, but it is generally believed that the high stress on peri-infarct tissue plays an important role. The principal components of the remodeling event include myocyte death, edema and inflammation, followed by fibroblast infiltration and collagen deposition, and, finally, scar formation. Pfeffer, et al., Circulation, 81:1161-1172 (1990). The principal component of the scar is collagen. Immediately after a myocardial infarction, the injury area expands, followed by regional dilation and thinning of the infarct zone. Kehat, et al., Circulation, 122:2727-2735 (2010). In other areas, remote regions experience hypertrophy (thickening), resulting in an overall enlargement of the left ventricle. Pfeffer, et al., Circulation, 81:1161-1172 (1990). Although the term cardiac remodeling was initially coined to describe changes in the heart following MI, it is clear that similar processes transpire after other types of injury such as with pressure overload (aortic valve stenosis, hypertension), inflammatory disease (myocarditis), idiopathic dilated cardiomyopathy, and volume overload (valvular regurgitation). Although the causes of these diseases are different, they share molecular, biochemical, and cellular events to collectively change the shape of the myocardium. Kehat, et al., Circulation, 122:2727-2735 (2010).

Myocardial infarction has profound effects on the general function of the heart. Ejection fraction, the amount of blood in the ventricle that is ejected with each stroke of the heart, decreases depending on the size of the infarction. The normal stroke volume, the amount of blood ejected from the ventricle with each heartbeat, is initially maintained despite the decrease in ejection fraction because of compensatory responses. The compensatory responses increase the stress in the ventricular wall because of the extra pressure and volume applied. The increase in stress can cause complications such as aneurysms and rupture.

Various procedures can reduce damage to the heart following MI. One approach focuses on reopening blocked arties. Some of the procedures include including mechanical and therapeutic agent application procedures. An example of a mechanical procedure is balloon angioplasty with stenting. An example of a therapeutic agent application includes the administration of a thrombolytic agent, such as urokinase. Systemic drugs, such as ACE-inhibitors and Beta-blockers, may be effective in reducing cardiac load post-MI, although a significant portion of the population that experiences a major MI ultimately develop heart failure.

Some research has focused on the use of stem cell therapy for the regeneration of the myocardium post MI. Patients who receive stem cell treatment by left ventricular intramyocardial implantation of stem cells derived from their own bone marrow after a myocardial infarction show improvements in left ventricular ejection fraction and end-diastolic volume, which is not seen with placebo. The larger the initial infarct size, the greater the effect of the infusion. Clinical trials of progenitor cell infusion as a treatment approach has also been conducted (Schachinger, et al., N. Engl. J. Med., 355(12): 1210-21 (2006)). Other approaches include biomaterial and tissue engineering approaches. One approach uses polymeric left ventricular restraints in the prevention of heart failure. A second approach utilizes in vitro engineered cardiac tissue, which is subsequently implanted in vivo. Still another approach entails injecting cells and/or a scaffold into the myocardium to create in situ engineered cardiac tissue (Christman, et al., J. Am. Coll. Cardiol., 48(5): (2006)). A variant of this approach is the injection of cells to produce factors that will help preserve myocardium post MI, for instance, by preventing the cardiomyocytes from undergoing apoptosis (Bose, et al., Cardiovasc. Drugs Ther., 21(4):253-6 (2007); and Nikolaidis, et al., Circulation, 109(8):962-5 (2004)).

Despite the numerous proposed theories, there has been little reduction to practice and demonstration of efficacy. An effective therapy has not yet been established, in part due to a lack of appropriate cells that will survive and develop the requisite mechanical properties, especially once scar formation is initiated or there are large areas of necrotic tissue.

It is therefore an object of the present invention to provide compositions and methods for delaying, attenuating or preventing adverse cardiac remodeling following cardiac injury.

### SUMMARY OF THE INVENTION

Compositions for delaying, attenuating or preventing adverse cardiac remodeling following cardiac injury contain fibroblast cells in an effective amount to delay, attenuate or prevent adverse cardiac remodeling following cardiac injury. In one embodiment, the fibroblast cells are autologous. In another embodiment, the fibroblast cells are allogeneic cells, obtained from a screened donor. These cells are obtained by biopsy, preferably from the patient, then cultured and proliferated prior to use. It has been discovered that certain subpopulations of these cells are even better suited for repair or regeneration of tissue, the cells exhibiting properties similar to stem cells or multipotent cells.

In a preferred embodiment, the cells are injected into the myocardial tissue following cardiac injury. Preferably the cells are administered in small doses in multiple areas of the infarcted tissue or adjacent to the infarcted tissue. In rat studies, dosages of one million cells were administered in a volume of 80 microliters, injected in volumes of 20 microliters.

### BRIEF DESCRIPTION OF THE DRAWINGS

FIG. 1 provides a summary of prerequisites for cell-based therapies which are particularly important when cells are delivered intravascularly.

FIG. 2 is a schematic of the protocol used in the mouse study.

FIG. 3A is a bar graph showing fractional shortening at baseline, in the day 7 injection group, the control group and the autologous fibroblast treatment group. FIG. 3B is a bar graph showing percent change in fractional shortening in the control and the autologous fibroblast treatment group.

FIG. 4A is a bar graph showing ejection fraction at baseline, in the day 7 injection group, the control group and the autologous fibroblast treatment group. FIG. 4B is a bar graph showing percent change in ejection fraction in the control and the autologous fibroblast treatment group. FIG. 4C is a bar graph showing the percent change in left ventricular area in diastole at the papillary muscle level in the control and the autologous fibroblast treatment group. FIG. 4D is a graph showing infarction size in control animals and the autologous fibroblast treatment group.

### DETAILED DESCRIPTION OF THE INVENTION

### I. Definitions

The phrase “cardiac remodeling” refers to the changes in size, shape, and associated function of the heart after injury to the left and right ventricle and/or right and left atrium.

The term “Autologous” is used herein to refer to the donor and recipient of the fibroblast cells being the same.

The term “Allogeneic” is used herein to refer to the donor and recipient of the fibroblast cells being different individuals of the same species.

The term “cardiac injury” as used herein includes any disease or condition that results in cardiac remodeling.

The phrase “ejection fraction” or “EF” means the portion of blood that is pumped out of a filled ventricle as the result of a heartbeat. It may be defined by the following formula:

$$\frac{LV End Diastolic Volume - LV End Systolic Volume \times 100}{LV End Diastolic Volume} \qquad$$

The term “fibroblasts” refers to specialized cells found in the body, for example, in the skin, that produce collagen and other extracellular matrix components to form connective tissues. These cells play critical roles in the development of human tissue.

The phrase “fractional shortening” as used herein refers to a measure of left ventricular function and may be determined by measuring the change in the diameter of the left ventricle between the contracted and relaxed states. The percent fractional shortening is calculated as the difference between the left ventricle end-diastolic diameter (LVd) and the left ventricle end-systolic diameter (LVs) divided by the left ventricle end-diastolic diameter (LVd):

$$\frac{LVd - LVs \times 100}{LVd}$$

### II. Compositions

A. Sources of Cells

The cells used in the method described herein can be autologous or allogeneic, preferably autologous. The autologous fibroblast cell therapy product is derived from the patient into whom the cells are to be implanted.

1. Autologous Dermal Fibroblasts

The autologous fibroblast cell therapy product is derived from the patient into whom the cells are to be implanted. The cell therapy product is composed of a suspension of autologous fibroblasts, grown from a biopsy of each individual's own skin using standard tissue culture procedures. The cell therapy product consists of expanded fibroblasts, formulated to the target cell concentration and cryopreserved in cryovials, called Bulk Drug Substance—Cryovial. The final cell therapy product consists of thawed Bulk Cell therapy product—Cryovial cells that are thawed, washed and prepared for patient injection.

The cells in the formulation display typical fibroblast morphologies when growing in cultured monolayers. Specifically, cells may display an elongated, fusiform or spindle appearance with slender extensions, or cells may appear as larger, flattened stellate cells which may have cytoplasmic leading edges. A mixture of these morphologies may also be observed. The cells express proteins characteristic of normal fibroblasts including the fibroblast-specific marker, CD90 (Thy-1), a 35 kDa cell-surface glycoprotein, and the extracellular matrix protein, collagen.

2. Allogeneic Fibroblasts

Fibroblasts are obtained from a screened donor(s) using similar methods as described above. In this embodiment, a screened donor provides tissue for expansion of fibroblasts and creation of a master cell bank (MCB). After appropriate tests are conducted on the MCB, cells expanded from the master bank are used to create a working cell bank (WCB), which is in turn expanded for manufacture of conditioned media for use in the formulation of the allogeneic topical product. The manufacturing process is similar to the autologous process, has the same applications and all final formulations are within the same concentration ranges.

3. Fibroblast Subpopulations

Somatic cells transfected with retroviral vectors that express OCT4, SOX2, KLF4 and cMYC to generate induced pluripotent stem cells (“iPSCs”) express the same pluripotency markers as control H9 ESCs. Reprogrammed cells possess a normal karyotype, differentiate into beating cardiomyocytes in vitro and differentiate into representatives of all three germ layers in vivo.

A subpopulation of human dermal fibroblasts that express the pluripotency marker stage specific embryonic antigen 3 (SSEA3) demonstrates enhanced iPSC generation efficiency as described by Bryne, et al., PLoS One, 4(9):e7118 (2009). SSEA3-positive and SSEA3-negative populations were transduced with the same retroviral vectors, under identical experimental conditions, and seeded onto inactivated mouse embryonic fibroblasts (MEFs). After three weeks of culture under standard hESC conditions, plates were examined in a double-blind analysis by three independent hESC biologists for iPSC colony formation. Colonies with iPSC morphology were picked and expanded. All three biological replicates with the transduced SSEA3-negative cells formed many large background colonies (10-27 per replicate) but no iPSC colonies emerged; in contrast, all three biological replicates with the transduced SSEA3-positive cells resulted in the formation of iPSC colonies (4-5 per replicate) but very few large background colonies (0-1 per replicate). Further characterization of the cell lines derived from the iPSC-like colonies showed that they possessed hESC-like morphology, growing as flat colonies with large nucleo-cytoplasmic ratios, defined borders and prominent nucleoli. When five lines were further expanded and characterized, all demonstrated expression of key pluripotency markers expressed by hESCs, which included alkaline phosphatase, Nanog, SSEA3, SSEA4, TRA160 and TRA181. The SSEA3-selected iPSCs also demonstrated a normal male karyotype (46, XY), the ability to differentiate into functional beating cardiomyocytes in vitro and differentiate into representatives of all three germ layers in vivo. Since no iPSC colony formation or line derivation from the transduced SSEA3-negative cells was observed, this indicates that these cells possess significantly lower or even no reprogramming potential relative to the SSEA3-expressing cells. Additionally, a 10-fold enrichment of primary fibroblasts that strongly express SSEA3 results in a significantly greater efficiency (8-fold increase) of iPSC line derivation compared to the control derivation rate (p<0.05). The SSEA3-positive cells appeared indistinguishable, morphologically, from the SSEA3-negative fibroblasts; furthermore, expression of the SSEA3 antigen is not considered a marker of other cell types such as mesenchymal or epidermal adult stem cells.

A rare subpopulation of SSEA3 expressing cells was isolated that exists in the dermis of adult human skin. These SSEA3-expressing cells undergo a significant increase in cell number in response to injury, indicating a role in regeneration. These SSEA3-expressing regeneration-associated (SERA) cells were derived through primary cell culture, purified by fluorescence activated cell sorting (FACS) and characterized. The SERA cells demonstrated a global transcriptional state most similar to bone marrow and fat derived mesenchymal stem cells (MSCs) and the highest expressing SSEA3 expressing cells co-expressed CD105. However, these cells cannot differentiate into adipocytes, osteoblasts or chondrocytes. These cells represent a preferred population for use in cardiac repair or regeneration.

B. Additional Therapeutic, Prophylactic and Diagnostic Agents

Pharmaceutical agents which may be administered together with the fibroblasts include, but are not limited to, small molecule drugs, oligonucleotides, peptides and proteins which can inhibit the negative remodeling response, growth factors, or compounds which stimulate angiogenesis or regeneration of cardiac tissue. Cell survival promoting factors can also be used to increase the survivability of autologous and allogeneic implanted cells.

The agent is preferably an agent that benefits a damaged blood vessel or an infarcted area, for example, by creating new cells or new cell components or triggering a repair mechanism. Suitable agents include, but are not limited to, growth factors (e.g., vascular endothelial growth factor (VEGF), fibroblast growth factor (FGF), platelet-derived growth factor (PDGF), insulin-like growth factor (IGF), hepatocyte growth factor (HGF), basic fibroblast growth factor (bFGF), acidic fibroblast growth factor (aFGF), placental growth factor (P1GF), granulocyte colony-stimulating factor (G-CSF)), cellular components, and cytokines.

Agents to induce regression or slow progress of an atherosclerotic plaque can also be administered with the fibroblasts. Examples include apolipoprotein A1 (Apo A1) or a mutant or mimic form of Apo A1, or a molecule mimicking the cholesterol transporting capacity of ApoA1.

Other therapeutics that could be administered with the fibroblasts include HDL mimetics, for example, cyclodestrin; anti-inflammatory agents, for example, clobetasol, dexamethasone, prednisone, aspirin and cordisone; and anti-proliferative agents, for example, taxol, everolomus, sirolomus, and doxorubicin to reduce scar tissue formation.

C. Carriers for Fibroblasts

The fibroblasts can be suspended in any sterile pharmaceutically acceptable carrier used for delivering cells into the myocardium, or seeded onto devices for implantation into the damaged tissue.

Preferred excipients for injection of a cell suspension include sterile saline, phosphate buffered saline, and other sterile isotonic excipients suitable for delivery of cells. The most preferred carrier is DMEM, the FDA approved carrier for LAVIV® fibroblasts for injection.

Various biomaterials are known which also can be used for cell delivery to the myocardium. Biomaterials control of the cellular microenvironment.

In one embodiment, the cells are suspended in a hydrogel material such as gelatin, fibrin, collagen, or alginates which form gels or three dimensional scaffolds. Suitable materials are described in U.S. Pat. No. 6,730,298 to Griffith-Cima, et al. Alginate is a natural polysaccharide derived from brown seaweed. Alginate is used in gel and 3D sponge form for cell delivery to the infarcted heart. Rowley, et al., describes covalently modified alginate polysaccharides with RGD-containing cell adhesion ligands (Rowley, et al., Biomaterials, 20(1):45-53 (1999)) which can be used for cell delivery.

Collagen is the most abundant protein in mammals. It is also known as the primary component of connective tissue. Collagen has been used successfully in cardiac applications for cell delivery and contraction. Like alginate, collagen comes in many forms; gels or 3D sponges are most common (Eschenhagen, et al., FASEB J., 11(8):683-94 (1997); Simpson, et al., Stem Cells, 25(9):2350-7 (2007) and Kofidis, et al., Eur. J. Cardiothorac. Surg., 22(2):238-43 (2002). Collagen has several properties that demonstrate its potential as a scaffold including cell attachment, cell proliferation, high hydrophilicity, and degradability. Veritas™ is an example of a 3D collagen matrix that can be utilized to deliver cells to the heart.

Fibrin is a naturally occurring matrix, created during the wound healing process and serves as a provisional matrix for cell attachment and migration. Fibrin gels have been used to deliver cells to the infarcted myocardium in several studies (Christman, et al., J. Am. Coll. Cardiol., 44(3):654-60 (2004) and Wei, et al., Biomaterials, 29(26):3547-56 (2008). Fibrin microthreads, a form of fibrin shaped like a suture and having significantly higher tensile strength than other forms of fibrin including fibrin gels and glue can also be used to deliver fibroblasts to the myocardium. Megan, et al., J. Mater Res. A, 96(2):301-312 (2011) describes cell-seeded fibrin microthreads which can serve as a platform to improve localized delivery and engraftment of viable cells to damaged tissue.

Other materials such as hyaluronic acid can also be used. See, for example, U.S. Pat. No. 8,193,340 to Kim, et al. Hyaluronic acid is dissolved in an aqueous sodium hydroxide solution; an epoxy-based crosslinking agent is added to the resultant aqueous sodium hydroxide solution in which the hyaluronic acid is dissolved, homogenizing the hyaluronic acid solution; the homogenized hyaluronic acid solution is gelled and washed with ultrapure water, swelling the washed hyaluronic acid hydrogel to attain porosity, and the hyaluronic acid hydrogel freeze dried to obtain a porous hyaluronic acid sponge. U.S. Pat. No. 8,178,663 to Bellini, et al., describes esters of hyaluronic acid which can be crosslinked by photocuring, which are also useful.

U.S. Pat. No. 8,192,760 to Hossainy, et al., describes a method to make compositions for delivery of drugs or cells using silk proteins. In one embodiment, a first component can include a first functionalized polymer, a second component can include a second functionalized polymer and a third component can include silk protein or constituents thereof.

Chitosan mixtures can also be formed into hydrogels for delivery of cells. See, for example, U.S. Pat. No. 8,153,612 to Ben-Shalom, et al. This patent describes a chitosan composition which forms a hydrogel at near physiological pH and 37° C., comprising at least one type of chitosan having a degree of acetylation in the range of from about 30% to about 60%, and at least one type of chitosan having a degree of deacetylation of at least about 70%, preferably with molecular weights of from 10-4000 kDa and from 200-20000 Da.

Still other materials are formed of proteins and polyglycans, as described by U.S. Pat. Nos. 8,053,423 and 7,799,767 to Lamberti, et al.

In another embodiment, the cells are attached, prior to or at the time of implantation, to a fibrous scaffold. These generally require open heart surgery to implant, however. Examples include:

Biodegradable cardiovascular patches may be used for vascular patch grafting, (pulmonary artery augmentation), for intracardiac patching, and for patch closure after endarterectomy. Examples of similar state of the art (non-degradable) patch materials include Sulzer Vascutek FLUOROPASSIC® patches and fabrics (Sulzer Carbomedics Inc., Austin, Tex.). See also U.S. Pat. No. 7,396,537 to Krupnick, et al. Cardiovascular patches can be fabricated according to the methods and procedures described in U.S. Pat. Nos. 5,716,395; 5,100,422, 5,104,400; and 5,700,287; and by Malm, et al., Eur. Surg. Res., 26:298-308 (1994). Tissue engineering scaffolds formed from woven or non-woven fibers are described in U.S. Pat. Nos. 5,770,417, 5,770,193, 5,759,830, 5,736,372, 5,716,404, 5,514,378, 5,399,665, and 5,041,138.

Other devices that may be utilized with these cells, or adapted for use in the applications described herein, include the following surgical devices.

Biodegradable surgical meshes may be used in general surgery. Examples of such state of the art meshes include the Brennen Biosynthetic Surgical Mesh Matrix (Brennan Medical, St. Paul, Minn.), GORE-TEX® Patches (Gore, Flagstaff, Ariz.), and SEPRAMESH® (Genzyme Corporation, Mass.). Surgical meshes can be fabricated according to the methods and procedures described by Bupta, “Medical textile structures: an overview” Medical Plastics and Biomaterials, pp. 16-30 (January/February 1998) and by methods described in U.S. Pat. Nos. 5,843,084; 5,836,961; 5,817,123; 5,747,390; 5,736,372; 5,679,723; 5,634,931; 5,626,611; 5,593,441; 5,578,046; 5,516,565; 5,397,332; 5,393,594; 5,368,602; 5,252,701; 4,838,884; 4,655,221; 4,633,873; 4,441,496; 4,052,988; 3,875,937; 3,797,499; and 3,739,773.

Biodegradable repair patches may be used in general surgery. For example, these patches may be used for pericardial closures, to reinforce staple lines and long incisions, and other soft tissue repair, reinforcement, and reconstruction. Examples of such state of the art patches include the TISSUEGUARD® product (Bio-Vascular Inc., St. Paul, Minn.). Repair patches can be fabricated according to the methods and procedures described in U.S. Pat. Nos. 5,858,505; 5,795,584; 5,634,931; 5,614,284; 5,702,409; 5,690,675; 5,433,996; 5,326,355; 5,147,387; 4,052,988, and 3,875,937.

### III. Method of Making and Using

Skin tissue (dermis and epidermis layers) is biopsied from a subject's post-auricular area. The starting material is composed of three 3-mm punch skin biopsies collected using standard aseptic practices. The biopsies are collected by the treating physician, placed into a vial containing sterile phosphate buffered saline (PBS). The biopsies are shipped in a 2-8° C. refrigerated shipper back to the manufacturing facility.

After arrival at the manufacturing facility, the biopsy is inspected and, upon acceptance, transferred directly to the manufacturing area. Upon initiation of the process, the biopsy tissue is then washed prior to enzymatic digestion. After washing, a Liberase Digestive Enzyme Solution is added without mincing, and the biopsy tissue is incubated at 37.0±2° C. for one hour. Time of biopsy tissue digestion is a critical process parameter that can affect the viability and growth rate of cells in culture. Liberase is a collagenase/neutral protease enzyme cocktail obtained unformulated from Roche Diagnostics Corp. (Indianapolis, Ind.). Alternatively, other commercially available collagenases may be used, such as Serva Collagenase NB6 (Helidelburg, Germany). After digestion, Initiation Growth Media (IMDM, GA, 10% Fetal Bovine Serum (FBS)) is added to neutralize the enzyme, cells are pelleted by centrifugation and resuspended in 5.0 mL Initiation Growth Media. Alternatively, centrifugation is not performed, with full inactivation of the enzyme occurring by the addition of Initiation Growth Media only. Initiation Growth Media is added prior to seeding of the cell suspension into a T-175 cell culture flask for initiation of cell growth and expansion. A T-75, T-150, T-185 or T-225 flask can be used in place of the T-75 flask.

Cells are incubated at 37±2.0° C. with 5.0±1.0% CO₂ and fed with fresh Complete Growth Media every three to five days. All feeds in the process are performed by removing half of the Complete Growth Media and replacing the same volume with fresh media. Alternatively, full feeds can be performed. Cells should not remain in the T-175 flask greater than 30 days prior to passaging. Confluence is monitored throughout the process to ensure adequate seeding densities during culture splitting. When cell confluence is greater than or equal to 40% in the T-175 flask, they are passaged by removing the spent media, washing the cells, and treating with Trypsin-EDTA to release adherent cells in the flask into the solution. Cells are then trypsinized and seeded into a T-500 flask for continued cell expansion. Alternately, one or two T-300 flasks, One Layer Cell Stack (1 CS), One Layer Cell Factory (1 CF) or a Two Layer Cell Stack (2 CS) can be used in place of the T-500 Flask.

Morphology is evaluated at each passage and prior to harvest to monitor the culture purity throughout the culture purity throughout the process. Morphology is evaluated by comparing the observed sample with visual standards for morphology examination of cell cultures. The cells display typical fibroblast morphologies when growing in cultured monolayers. Cells may display either an elongated, fusiform or spindle appearance with slender extensions, or appear as larger, flattened stellate cells which may have cytoplasmic leading edges. A mixture of these morphologies may also be observed. Fibroblasts in less confluent areas can be similarly shaped, but randomly oriented. The presence of keratinocytes in cell cultures is also evaluated. Keratinocytes appear round and irregularly shaped and, at higher confluence, they appear organized in a cobblestone formation. At lower confluence, keratinocytes are observable in small colonies.

Cells are incubated at 37±2.0° C. with 5.0±1.0% CO₂ and fed every three to five days in the T-500 flask and every five to seven days in the ten layer cell stack (10 CS). Cells should not remain in the T-500 flask for more than 10 days prior to passaging. Quality Control (QC) release testing for safety of the Bulk Drug Substance includes sterility and endotoxin testing. When cell confluence in the T-500 flask is >95%, cells are passaged to a 10 CS culture vessel. Alternately, two Five Layer Cell Stacks (5 CS) or a 10 Layer Cell Factory (10 CF) can be used in place of the 10 CS. Passage to the 10 CS is performed by removing the spent media, washing the cells, and treating with Trypsin-EDTA to release adherent cells in the flask into the solution. Cells are then transferred to the 10 CS. Additional Complete Growth Media is added to neutralize the trypsin and the cells from the T-500 flask are pipetted into a 2 L bottle containing fresh Complete Growth Media. The contents of the 2 L bottle are transferred into the 10 CS and seeded across all layers. Cells are then incubated at 37±2.0° C. with 5.0±1.0% CO₂ and fed with fresh Complete Growth Media every five to seven days. Cells should not remain in the 10 CS for more than 20 days prior to passaging. No protein free medium is used in the expansion process at this time. The cryopreservative is the first protein-free medium used in the process. The FBS used in the media is provided with certificate of traceability from a non-TSE/BSE country ensure free of immunogenic proteins. It is also extensively virus tested. Primary Harvest When cell confluence in the 10 CS is 95% or more, cells are harvested. Harvesting is performed by removing the spent media, washing the cells, treating with Trypsin-EDTA to release adherent cells into the solution, and adding additional Complete Growth Media to neutralize the trypsin. Cells are collected by centrifugation, resuspended, and in-process QC testing performed to determine total viable cell count and cell viability.

For treatment of nasolabial folds, the total cell count must be 3.4×10⁸ cells and viability 85% or higher. Alternatively, total cell yields for other indications can range from 3.4×10⁸ to 1×10⁹ cells. Cell count and viability at harvest are critical parameters to ensure adequate quantities of viable cells for formulation of the Drug Substance. If total viable cell count is sufficient for the intended treatment, an aliquot of cells and spent media are tested for mycoplasma contamination. Mycoplasma testing is performed. Harvested cells are formulated and cryopreserved.

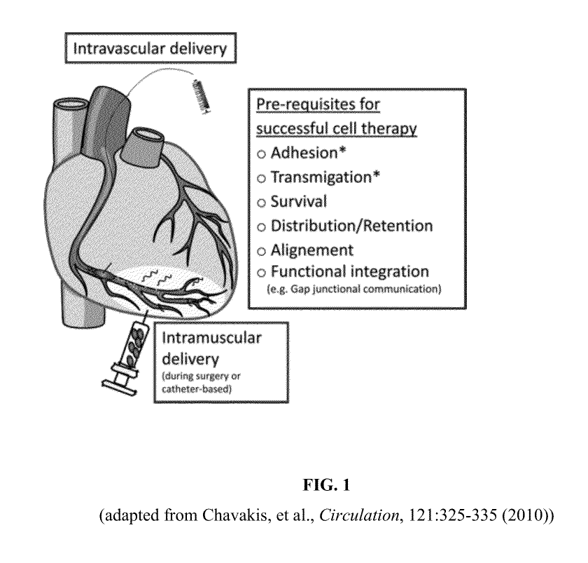

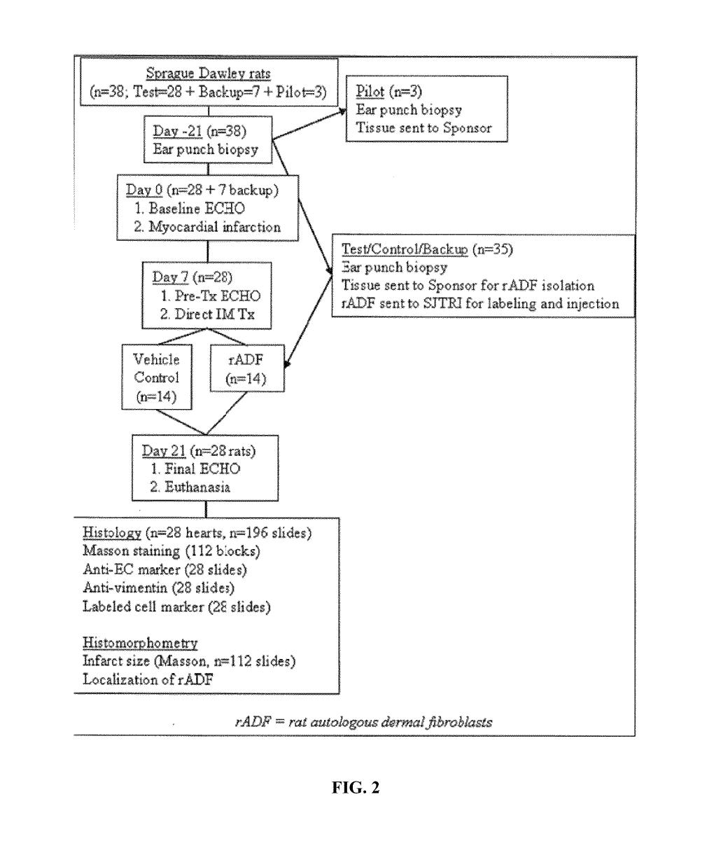

If additional cells are required after receiving cell count results from the primary 10 CS harvest, an additional passage into multiple cell stacks (up to four 10 CS) is performed (Step 5a in FIG. 1). For additional passaging, cells from the primary harvest are added to a 2 L media bottle containing fresh Complete Growth Media. Resuspended cells are added to multiple cell stacks and incubated at 37±2.0° C. with 5.0±1.0% CO₂. The cell stacks are fed and harvested as described above, except cell confluence must be 80% or higher prior to cell harvest. The harvest procedure is the same as described for the primary harvest above. A mycoplasma sample from cells and spent media is collected, and cell count and viability performed as described for the primary harvest above.

The method decreases or eliminates immunogenic proteins be avoiding their introduction from animal-sourced reagents. Trypsin and FBS are only animal sources reagents, FBS is only bovine; trypsin is porcine. To reduce process residuals, cells are cryopreserved in protein-free freeze media, then thawed and washed prior to prepping the final injection to further reduce remaining residuals.

If additional Drug Substance is needed after the harvest and cryopreservation of cells from additional passaging is complete (Step 5a in FIG. 1), aliquots of frozen Drug Substance—Cryovial are thawed and used to seed 5 CS or 10 CS culture vessels (Step 7a in FIG. 1). Alternatively, a four layer cell factory (4 CF), two 4 CF, or two 5 CS can be used in place of a 5 CS or 10 CS. A frozen cryovial(s) of cells is thawed, washed, added to a 2 L media bottle containing fresh Complete Growth Media and cultured, harvested and cryopreserved as described above. The cell suspension is added Cell confluence must be 80% or more prior to cell harvest.

C. Preparation of Cell Suspension

At the completion of culture expansion, the cells are harvested and washed, then formulated to contain 1.0-2.7×10⁷ cells/mL, with a target of 2.2×10⁷ cells/mL. Alternatively, the target can be adjusted within the formulation range to accommodate different indication doses. The Drug Substance consists of a population of viable, autologous human fibroblast cells suspended in a cryopreservation medium consisting of Iscove's Modified Dulbecco's Medium (IMDM) and Profreeze-CDM™ (Lonza, Walkerville, Md.) plus 7.5% dimethyl sulfoxide (DMSO). Alternatively, a lower DMSO concentration may be used in place of 7.5% or CryoStor™ CS5 or CryoStor™ CS10 (BioLife Solutions, Bothell, Wash.) may be used in place of IMDM/Profreeze/DMSO. The freezing process consists of a control rate freezing step to the following ramp program:

### STEP 1: Wait at 4.0° C.

STEP 2: 1.0° C./minC/m to −4.0° C. (sample probe)
STEP 3: 25.0° C./minC/m to −40° C. (chamber probe)
STEP 4: 10.0° C./minC/m to −12.0° C. (chamber probe)
STEP 5: 1.0° C./minC/m to −40° C. (chamber probe)
STEP 6: 10.0° C./minC/m to −90° C. (chamber probe)

### STEP 7: End

After completion of the controlled rate freezing step, Bulk Drug

Substance vials are transferred to a cryogenic freezer for storage in the vapor phase. After cryogenic freezing, the Drug Substance is submitted for Quality Control testing. Drug Substance specifications also include cell count and cell viability testing performed prior to cryopreservation and performed again for Drug Substance—Cryovial. Viability of the cells must be 85% or higher for product release. Cell count and viability are conducted using an automated cell counting system (Guava Technologies), which utilizes a combination of permeable and impermeable fluorescent, DNA-intercalating dyes for the detection and differentiation of live and dead cells. Alternatively, a manual cell counting assay employing the trypan blue exclusion method may be used in place of the automated cell method above. Alternatively, other automated cell counting systems may be used to perform the cell count and viability method, including Cedex (Roche Innovatis AG, Bielefield, Germany), ViaCell™ (Beckman Coulter, Brea, Calif.), NuceloCounter™ (New Brunswick Scientific, Edison, N.J.), Countless® (Invitrogen, division of Life Technologies, Carlsbad, Calif.), or Cellometer® (Nexcelom Biosciences, Lawrence, Mass.). Drug Substance—Cryovial samples must meet a cell count specification of 1.0-2.7×10⁷ cells/mL prior to release. Sterility and endotoxin testing are also conducted during release testing.

In addition to cell count and viability, purity/identity of the Drug Substance is performed and must confirm the suspension contains 98% or more fibroblasts. The usual cell contaminants include keratinocytes. The purity/identify assay employs fluorescent-tagged antibodies against CD90 and CD104 (cell surface markers for fibroblast and keratinocyte cells, respectively) to quantify the percent purity of a fibroblast cell population. CD90 (Thy-1) is a 35 kDa cell-surface glycoprotein. Antibodies against CD90 protein have been shown to exhibit high specificity to human fibroblast cells. CD104, integrin 134 chain, is a 205 kDa transmembrane glycoprotein which associates with integrin α6 chain (CD49f) to form the α6/β4 complex. This complex has been shown to act as a molecular marker for keratinocyte cells (Adams and Watt 1991).

Antibodies to CD104 protein bind to 100% of human keratinocyte cells.

Cell count and viability is determined by incubating the samples with Viacount Dye Reagent and analyzing samples using the Guava PCA system. The reagent is composed of two dyes, a membrane-permeable dye which stains all nucleated cells, and a membrane-impermeable dye which stains only damaged or dying cells. The use of this dye combination enables the Guava PCA system to estimate the total number of cells present in the sample, and to determine which cells are viable, apoptotic, or dead. The method was custom developed specifically for use in determining purity/identity of autologous cultured fibroblasts.

Methods of Administration

The fibroblast cells described above alone or in combination with additional bioactive agents are delivered into and/or adjacent the infarct zone of the myocardium or to damaged or diseased myocardial tissue.

Four major techniques for cell administration include (1) intramyocardial, which include epicardial and transendocardial; (2) intracoronary; (3) transvenous coronary sinus; and (4) intravenous. Reviewed in Dib, et al., J. Cardiovasc. Trans'. Res., 4(2):177-181 (2011).

i. Intramyocardial Administration

Intramyocardial administration involves injection directly into the myocardium. Injections are most frequently made into the left ventricle by a direct epicardial approach or using a catheter-based transendocardial approach. Epicardial injection is considered the most reliable method of delivery, which also results in higher cell retention within the myocardium. In previous clinical trials of epicardial injection, cell transplant was performed using a minimally invasive surgical approach via a left anterior mini-thoracotomy or combined with coronary artery bypass graft or other open heart procedures. Surgical exposure of the heart provides direct access and visualization of the epicardium. Location of the injection sites are identified prior to surgery using non-invasive methods including nuclear imaging and echocardiography. During surgery, injection sites are located by direct visualization and therapy is administered to the external surface of the heart via a standard syringe. Injections can be made into a beating or arrested heart. Dib, et al., J. Cardiovasc. Transl. Res., 4(2):177-181 (2011).

For direct injection, a small bolus of selected genetic material and/or undifferentiated or differentiated contractile cells can be loaded into a micro-syringe, e.g., a 100 μL Hamilton syringe, and applied directly from the outside of the heart.

Alternatively, the fibroblasts can be administered by transendocardial injection. Transendocardial injection utilizes a percutaneous catheter-based approach. For example, a catheter can be introduced from the femoral artery and steered into the left ventricle, which can be confirmed by fluoroscopy. The catheter can also be steered into the right ventricle. The catheter includes an elongated catheter body, suitably an insulative outer sheath which may be made of polyurethane, polytetrafluoroethylene, silicone, or any other acceptable biocompatible polymer, and a standard lumen extending there through for the length thereof, which communicates through to a hollow needle element. The catheter may be guided to the indicated location by being passed down a steerable or guidable catheter having an accommodating lumen, for example as disclosed in U.S. Pat. No. 5,030,204 (Badger et al.); or by means of a fixed configuration guide catheter such as illustrated in U.S. Pat. No. 5,104,393 (Isner et al.). Alternately, the catheter may be advanced to the desired location within the heart by means of a deflectable stylet, as disclosed in PCT Patent Application WO 93/04724, published Mar. 18, 1993, or by a deflectable guide wire as disclosed in U.S. Pat. No. 5,060,660 (Gambale et al.). In yet another embodiment, the needle element may be ordinarily retracted within a sheath at the time of guiding the catheter into the patient's heart. Once in the left (or right) ventricle, the tip of the catheter can be moved around the left ventricular wall as probe to measure the electrogram and to determine the location and extent of the infarct zone. This is a procedure known to one of skill in the art. Once the infarct zone is identified, the steering guide will be pulled out leaving the sheath at the site of infarction. The cell repopulation source and/or electrical stimulation device can then be sent down the lumen of the catheter and pushed into the myocardium. The catheter can then be retracted from the patient. There are varieties of catheters currently undergoing Phases I and II clinical trials which use either a fluoroscopic 2-dimensional (2D) guidance system or a 3-dimensional (3D) system. The Helix™ infusion catheter (BioCardia, Inc., South San Francisco, Calif.) and the MyoCath™ (Bioheart Inc., Sunrise, Fla.), are 2D systems. The Myostar™ Injection Catheter is combined with a 3D guidance system NOGA® XP (Biologics Delivery Systems, Diamond Bar, Calif.) Sherman, et al., Nature Clinical Practice. Cardiovascular Medicine, 3(Suppl. 1):558-560 (2006); Fuente, et al., American Heart Journal, 154:79 (2007); Amado, et al., PNAS, 102:11474-11479 (2005); Vale, et al., Circulation, 103:2138-2143 (2001).

ii. Intracoronary Administration

Intracoronary administration is the preferred technique following acute myocardial infarction and has been previously described extensively and reviewed in Dib, et al., J. Cardiovasc. Trans'. Res., 4(2):177-181 (2011). Intracoronary administration is similar to balloon angioplasty, and it is the most practiced technique of coronary cell transfer. Cells are injected through the delivery catheter at slow or high flow rates while maintaining coronary flow (non-occlusive) or interrupting it with balloon occlusion (“stop-flow” method). Dib, et al., Journal of the American College of Cardiology Cardiovascular Interventions, 3:265-275 (2010). In the case of non-occlusive angioplasty method, a balloon catheter or specialty catheters are used for the sub-selective injection in the coronary vessel. “Stop-flow” method uses a temporary balloon inflation to reduce cell loss due to speed of blood flow.

iii. Transvenous Coronary Sinus Delivery

The coronary sinus (CS) and coronary veins have been utilized in applications for several therapeutic interventions. The retrograde coronary sinus delivery method provides access to the target ischemic and infarcted regions of the heart. Details of this procedure have been previously described (Dib, et al., J. of the Am. Coll. of Cardiol. Cardiovas. Interven., 3:265-275 (2010); Pohl, et al., Cat. and Cardiovas. Interven., 62:323-330 (2004); Raake, et al., J. of the Am. Coll. of Cardiol., 44:1124-1129 (2004); and Degenfeld, et al., J. of the Am. Coll. of Cardiol., 42:1120-1128 (2003). A small number of preclinical and early clinical studies demonstrated safety and feasibility of delivering stem cells via the coronary sinus. Coronary sinus delivery is the preferred option in cases of severe subtotal stenosis of one or more coronary arteries or severe aortic stenosis. This approach provides safe and accurate access to most of the myocardium creating more homogenous delivery.

iv. Intravenous Delivery

The fibroblasts can be delivered to the myocardium systemically. Methods for systemic cell delivery are known in the art. Systemic cell delivery is very low risk and utilizes a standard intravenous infusion. It is the easiest to administer and the least invasive route of delivery. The low rate of cell homing, retention, and survival is one of the major limitations in current experimental and clinical studies with all different types of cells available. Particularly after intracoronary cell delivery, cells need to extravasate and transmigrate into the target tissue (FIG. 1). Direct injection of the cells into the myocardium or by perivascular delivery may provide an advantage in this respect. The understanding of homing mechanisms and tools can be used to improve survival and retention of systemically delivered fibroblasts. For example, the fibroblasts may be pretreated to stimulate adhesion, migration, survival, or differentiation.

The systemic delivery method depends heavily on cell homing signals to the area of injury following an acute myocardial infarction.

Patients to be Treated

The compositions are useful for delaying, attenuating or preventing adverse cardiac remodeling following cardiac injury. The injury is typically due to acute myocardial infarction (such as, for example transmural or ST segment elevation infarction) or induced injury (such as for example, heart surgery), but may be from a number of causes that result in increased pressure or volume overload (forms of strain) on the heart. Cardiac remodeling includes hypertrophy, thinning of the myocardium, scar formation of the myocardium, atrophy of the myocardium, heart failure progression and combinations thereof. Thus, patients with conditions which result in cardiac remodeling can benefit from the compositions disclosed herein. For example, patient with chronic hypertension, Kawasaki's disease, congenital heart disease with intracardiac shunting, and valvular heart disease may lead to remodeling. Additionally remodeling may stem from coronary artery bypass surgery, cardiac transplant and application of a mechanical support device, such as a left ventricular assist device (LVAD).

Detection of Myocardial Engraftment

Engraftment and repopulation can be monitored using various well-known imaging techniques such as scintigraphy, myocardial perfusion imaging, gated cardiac blood-pool imaging, first-pass ventriculography, right-to-left shunt detection, positron emission tomography, single photon emission computed tomography, magnetic resonance imaging, harmonic phase magnetic resonance imaging, echocardiography, and myocardial perfusion reserve imaging.

Cardiac scintigraphy evaluates myocardial perfusion and/or function to detect physiologic and anatomic abnormalities of the heart. The five major classes of cardiac scintigraphy include myocardial perfusion imaging, gated cardiac blood-pool imaging, first-pass cardiac imaging, myocardial infarction imaging, and right-to-left shunt evaluation (American College of Radiology Standard for the Performance of Cardiac Scintigraphy).

Myocardial perfusion imaging is used primarily to detect the presence, location, and extent of coronary artery disease by evaluating the physiologic significance or sequelae of known or suspected coronary artery stenosis, monitoring the effects of treatment of coronary artery disease, including revascularization and medical therapy. Myocardial perfusion imaging is also useful for detecting acute myocardial infarction and prognosis after infarction, for evaluating the viability of dysfunctional myocardium, for determining the risk of myocardial events, and for evaluating ventricular function.

An echocardiogram uses ultrasound is to examine the heart. In addition to providing single-dimension images, known as M-mode echo that allows accurate measurement of the heart chambers, the echocardiogram also offers two-dimensional (2-D) Echo and is capable of displaying a cross-sectional “slice” of the beating heart, including the chambers, valves and the major blood vessels that exit from the left and right ventricle.

Doppler assesses blood flow (direction and velocity). In contrast, the M-mode and 2-D Echo evaluates the size, thickness and movement of heart structures (chambers, valves, etc.). During the Doppler examination, the ultrasound beams will evaluate the flow of blood as it makes its way through and out of the heart. This information is presented visually on the monitor (as color images or grayscale tracings and also as a series of audible signals with a swishing or pulsating sound).

Echocardiography provides important information about, among other structures and functions, the size of the chambers of the heart, including the dimension or volume of the cavity and the thickness of the walls. The appearance of the walls may also help identify certain types of heart disease that predominantly involve the heart muscle. Pumping function of the heart can also be assessed by echocardiography. One can tell if the pumping power of the heart is normal or reduced to a mild or severe degree. This measure is known as an ejection fraction or EF. A normal EF is around 55 to 65%. Numbers below 45% usually represent some decrease in the pumping strength of the heart, while numbers below 30 to 35% are representative of an important decrease. Thus, echocardiography can assess the pumping ability of each chamber of the heart and also the movement of each visualized wall. The decreased movement, in turn, can be graded from mild to severe. In extreme cases, an area affected by a heart attack may have no movement (akinesia), or may even bulge in the opposite direction (dyskinesia). The latter is seen in patients with aneurysm of the left ventricle or LV.

Echocardiography identifies the structure, thickness and movement of each heart valve. It can help determine if the valve is normal, scarred from an infection or rheumatic fever, thickened, calcified, torn, etc. It can also assess the function of prosthetic or artificial heart valves. The additional use of Doppler helps to identify abnormal leakage across heart valves and determine their severity. Doppler is also very useful in diagnosing the presence and severity of valve stenosis or narrowing. Unlike echocardiography, Doppler follows the direction and velocity of blood flow rather than the movement of the valve leaflets or components. Thus, reversed blood direction is seen with leakages while increased forward velocity of flow with a characteristic pattern is noted with valve stenosis.

The volume status of blood vessels can also be monitored by echocardiography. Low blood pressure can occur in the setting of poor heart function but may also be seen when patients have a reduced volume of circulating blood (as seen with dehydration, blood loss, use of diuretics or “water pill”, etc.). In many cases, the diagnosis can be made on the basis of history, physical examination and blood tests. However, confusion may be caused when patients have a combination of problems. Echocardiography may help clarify the confusion. The inferior vena cava (the major vein that returns blood from the lower half of the body to the right atrium) is distended or increased in size in patients with heart failure and reduced in caliber when the blood volume is reduced. Echocardiography is useful in the diagnosis of fluid in the pericardium. It also determines when the problem is severe and potentially life threatening. Other diagnoses made by Doppler or echocardiography include congenital heart diseases, blood clots or tumors within the heart, active infection of the heart valves, abnormal elevation of pressure within the lungs, among others.

Myocardial perfusion reserve (MPR) quantifies the capacity of the circulatory response to a maximal increase in physiological demand (Siebert, et al., (2002) Proc. Intl. Soc. Mag. Reson. Med. vol. 10). MPR indicates the net circulatory consequence from coronary lesions and other vascular states, regardless of their morphological appearance, including the compensation by collateral circulation. Current perfusion acquisition methods now provide adequate temporal and spatial resolution, SNR, and first-pass contrast enhancement ratio. MPR imaging may provide quantitative, objective information to reduce variability in perfusion exam interpretation, and to document MR myocardial perfusion.

The present invention will be further understood by reference to the following non-limiting examples.

### EXAMPLES

### Example 1

### Myocardial Injection of Autologous Fibroblast Cell Suspension in Mice

Methods and Materials

FIG. 2 is a schematic of the protocol used in the mouse study.

On Day 7 after infarction, rats were given left lateral thoracotomy direct intra-myocardial injections of cell/control article. A total of four (4) injections of 20 μl each distributed in and around the infarcted region. Injections were performed using a glass Hamilton syringe with a 27G needle. A total of 1 million cells in 80 μl were delivered to the infarcted myocardium. Incisions were closed and the animals recovered.

Several Parameters were measured to determine changes in function.

Ejection fraction (EF) is the most commonly used parameter of left ventricular (LV) systolic function on clinical grounds. Dickstein, et al., Eur. Heart J., 29(19):2388-442 (2008). Following myocardial infarction, the ejection fraction (EF) is an indiscriminate predictor of both non-sudden cardiac death (NSCD) and sudden cardiac death (SCD). Bigger, et al., Circulation, 69:250-258 (1984); Marcus, et al., Am. J. Cardiol., 61:8-15 (1988); Mukharji, et al., Am. J. Cardiol., 54:31-36 (1984) and Califf, et al., Am. J. Cardiol., 67:454-459 (1991). Ventricular shortening fraction is the percentage change in diameter from diastole to systole measured using echocardiography. It is calculated from the internal systolic and diastolic dimensions. It is a measure of myocardial function.

Ejection fraction and fractional shortening were used as indices to determine the effect of fibroblast treatment on ventricular remodeling.

Following euthanasia, hearts were embedded in paraffin and cut into 3-4 mm thick transverse segments from apex to base. Thin sections (˜5 μM) were cut and cell nuclei were stained using DAPI. Slides were visualized using epifluorescent microscopy for cell nuclei (DAPI—blue) and labeled, injected fibroblasts (PKH67—yellow). All images were taken using a 20× objective.

Results

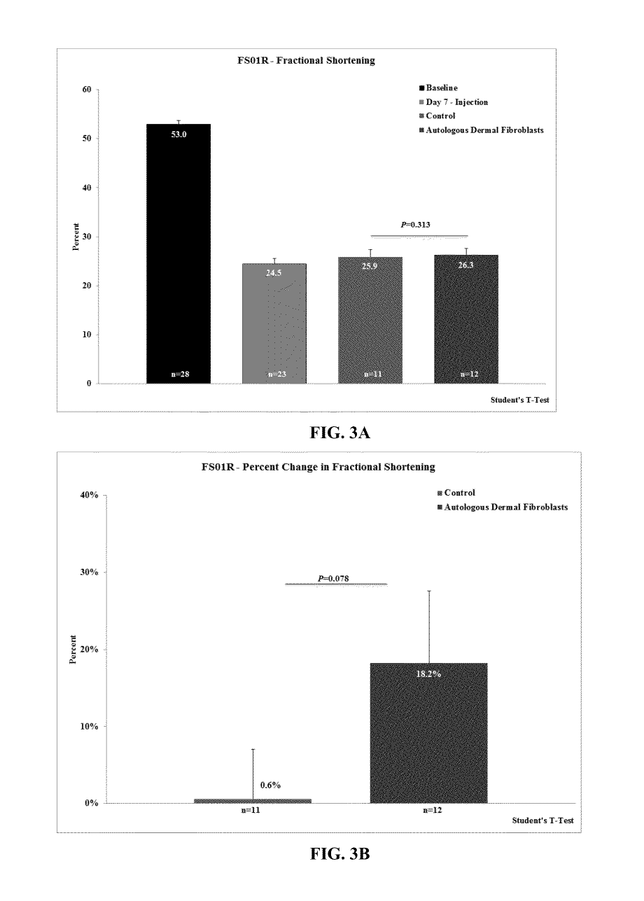

FIG. 3A is a bar graph showing fractional shortening at baseline, in the day 7 injection group, the control group and the autologous fibroblast treatment group. FIG. 3B is a bar graph showing percent change in fractional shortening in the control and the autologous fibroblast treatment group.

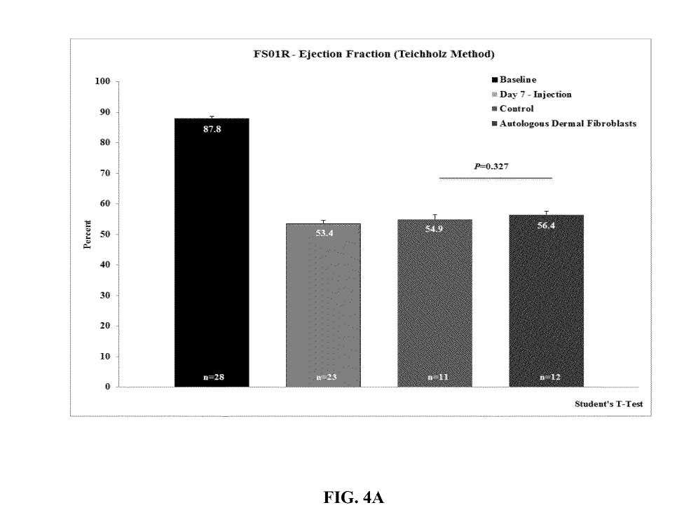

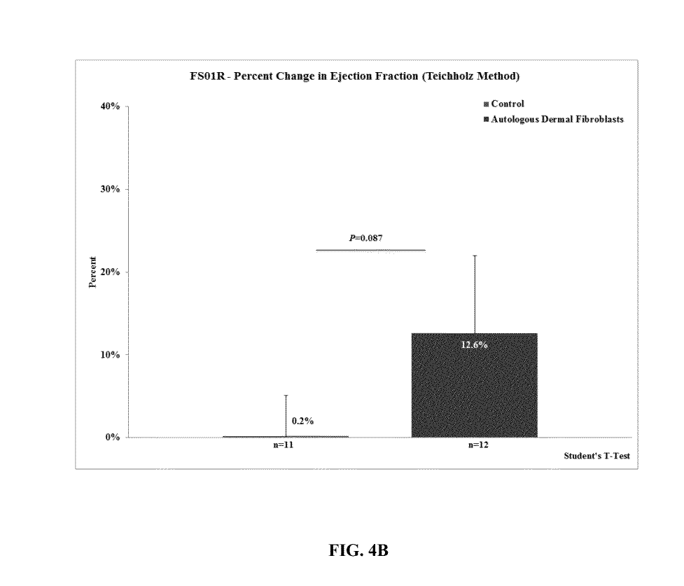

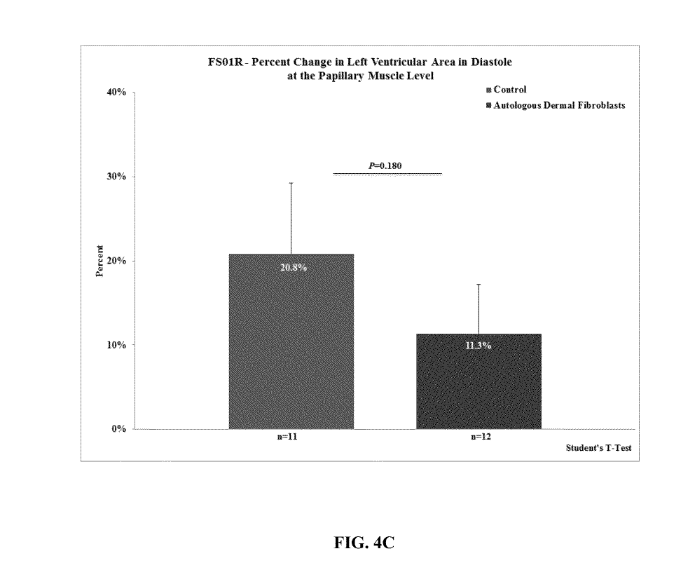

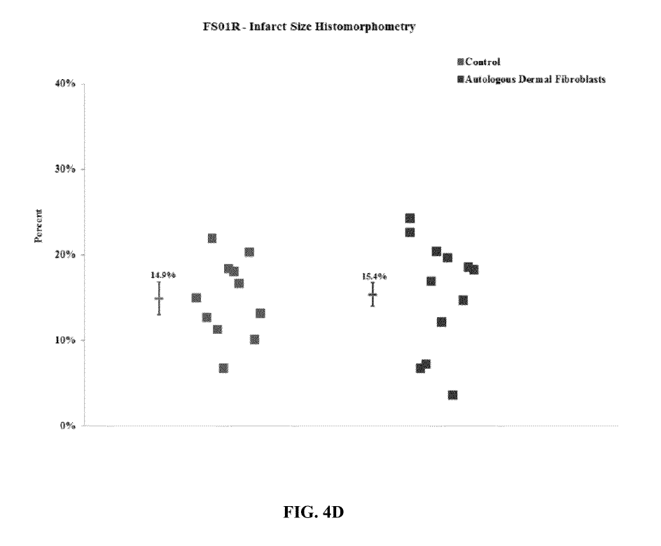

FIG. 4A is a bar graph showing ejection fraction at baseline, in the day 7 injection group, the control group and the autologous fibroblast treatment group. FIG. 4B is a bar graph showing percent change in ejection fraction in the control and the autologous fibroblast treatment group. FIG. 4C is a bar graph showing the percent change in left ventricular area in diastole at the papillary muscle level in the control and the autologous fibroblast treatment group. FIG. 4D shows the infarction size in control and autologous dermal fibroblast treatment groups.

As demonstrated by the graphs and Table 1, treatment of one week old myocardial infarcts with autologous dermal fibroblasts trended in an improvement in cardiac function (Fractional Shortening and Ejection Fraction). Cell treatment was associated with less ventricular dilatation along with less of a reduction in anterior and posterior wall thickness compared to controls. This improvement in cardiac function appears to be due to the ability of the cells to inhibit negative remodeling.

Histology studies showed PKH67 labeled fibroblasts in 8/12 injected animals. No labeled fibroblasts were seen in test animals 6138, 6151, 6152, 6155. In treatment animals 6143, 6153, 6156, and 6158 (4/12), multiple clusters of PKH67 labeled fibroblasts were seen. No PKH67 labeling was found in any control animals

Unless defined otherwise, all technical and scientific terms used herein have the same meanings as commonly understood by one of skill in the art to which the disclosed invention belongs. Publications cited herein and the materials for which they are cited are specifically incorporated by reference.

Those skilled in the art will recognize, or be able to ascertain using no more than routine experimentation, many equivalents to the specific embodiments of the invention described herein. Such equivalents are intended to be encompassed by the following claims.

TABLE 1
Echocardiographic Percent Change following Treatment
FSOIR - Echocardiographic Percent Change

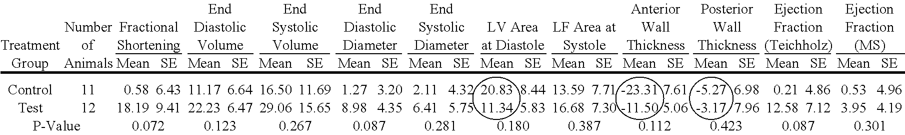

Echocardiographic assessment was performed at baseline (Day 0), prior to direct surgical injection (Day 7) and 2 weeks later at time of euthanasia (Day 21). Percent change for each animal was calculated using the following formula: ((Day 21 − Day 7)/Day 7) * 100. Table shows means ± SE and P-values from Student's T-Tests.

We claim:

1. A composition for delaying, attenuating or preventing adverse cardiac remodeling following cardiac injury consisting of isolated, cultured, proliferated fibroblast cells in a sterile pharmaceutically acceptable carrier in a dosage for administration to a site in need thereof to delay, attenuate or prevent adverse cardiac remodeling following cardiac injury, optionally comprising one or more therapeutic, prophylactic, or diagnostic agent.

2. The composition of claim 1 wherein the fibroblast cells are selected from the group of fibroblasts obtained by biopsy, cultured and proliferated, and subsets thereof having greater ability to differentiate.

3. The composition of claim 1 wherein the fibroblasts express SSEA3.

4. The composition of claim 1 wherein the cells are in a pharmaceutically acceptable carrier selected from the group consisting of sterile solutions, hydrogels, and implantable cell matrices or devices for implantation.

5. The composition of claim 4 wherein the cells are on a cardiovascular patch for vascular patch grafting, for pulmonary artery augmentation, for intracardiac patching, or for patch closure after endarterectomy.

6. A method for delaying, attenuating or preventing adverse cardiac remodeling following cardiac injury comprising administering isolated, cultured, proliferated fibroblast cells to a site in need thereof in a dosage effective to delay, attenuate or prevent adverse cardiac remodeling following cardiac injury.

7. The method of claim 6 wherein the fibroblast cells are selected from the group of fibroblasts obtained by biopsy, cultured and proliferated, and subsets thereof having greater ability to differentiate.

8. The method of claim 6 wherein the fibroblasts express SSEA3.

9. The method of claim 6 wherein the cells are in a pharmaceutically acceptable carrier selected from the group consisting of sterile solutions, hydrogels, and implantable cell matrices or devices for implantation.

10. The method of claim 9 wherein the cells are on a cardiovascular patch for vascular patch grafting, for pulmonary artery augmentation, for intracardiac patching, or for patch closure after endarterectomy.

11. The method of claim 6 wherein the cells are administered intramyocardial, epicardial or transendocardial.

12. The method of claim 6 wherein the cells are administered intracoronary.

13. The method of claim 6 wherein the cells are administered transvenous into the coronary sinus.

14. The method of claim 6 wherein the cells are administered intravenously.

15. The method of claim 6 wherein the cells are administered to a patient with cardiomyopathy resulting from a viral infection.
